(19) United States
(12) Patent Application Publication
(10) Pub. No.: US 2023/0201324 A1
(43) Pub. Date: Jun. 29, 2023

(54) Compositions and Methods for Treating Cocaine, Nicotine, and Methamphetamine Dependence

(71) Applicant: Babak Behnam, Clarksburg, MD (US)

(72) Inventors: Babak Behnam, Clarksburg, MD (US); Azim Akbarzadehkhiavi, Tehran (IR)

(73) Assignee: Babak Behnam, Clarksburg, MD (US)

(21) Appl. No.: 17/999,441

(22) PCT Filed: May 26, 2021
(86) PCT No.: PCT/US2021/034199

§ 371 Date: Nov. 21, 2022

Related U.S. Application Data
(60) Provisional application No. 63/030,401, filed on May 27, 2020.

(51) Int. Cl. A61K 39/00; A61K 47/64

(52) U.S. Cl. CPC A61K 39/0013; A61K 47/646; A61K 47/643; A61K 2039/6081; A61K 2039/55505

(57) ABSTRACT

This invention provides a composition of matter comprising a plurality of cocaine-succinyl-BSA, wherein the average ratio of cocaine-succinyl moieties to BSA is at least 7.0. This invention also provides a composition of matter comprising a plurality of nicotine-5-succinyl-BSA, wherein the average ratio of nicotine-5-succinyl moieties to BSA is at least 7.0. This invention further provides a composition of matter comprising a plurality of methamphetamine-5-succinyl-BSA, wherein the average ratio of methamphetamine-5-succinyl moieties to BSA is at least 7.0. This invention still further provides related pharmaceutical compositions, therapeutic methods, prophylactic methods, synthetic methods, and articles of manufacture.

This application claims the benefit of U.S. Provisional Application No. 63/030,401, filed May 27, 2020, the contents of which are incorporated herein by reference.

Throughout this application, various publications are cited. The disclosure of these publications is hereby incorporated by reference into this application to describe more fully the state of the art to which this invention pertains.

### BACKGROUND OF THE INVENTION

Cocaine

Cocaine is a strong and addictive stimulant. It is most frequently used as a recreational drug, and also has certain medical uses such as numbing and decreasing bleeding during nasal surgery. Globally, cocaine is the second most frequently used illicit drug (after cannabis), with 14-21 million people using it annually, and 4,300 use-related deaths in 2013.

Cocaine's mental effects include, for example, intense feelings of happiness, agitation, and loss of contact with reality. Its physical symptoms include, for example, accelerated heart rate, sweating, dilated pupils, and, at higher doses, high blood pressure and body temperature. Cocaine's effects begin immediately and last up to 90 minutes.

Risks of cocaine use include, for example, increased likelihood of stroke, myocardial infarction, lung problems (for those who smoke it), blood infections, and sudden cardiac death. Other such risks include, for example, decreased ability to feel pleasure, and physical exhaustion. At present, there are no drugs approved by the U.S. Food and Drug Administration to treat cocaine addiction.

Nicotine

Nicotine is a naturally occurring, highly addictive alkaloid stimulant. It constitutes approximately 0.6-3.0% of the dry weight of tobacco. An average cigarette yields about 2 mg of absorbed nicotine. Higher amounts can be more harmful.

Nicotine dependence involves tolerance, sensitization, physical dependence, and psychological dependence. Nicotine withdrawal symptoms include depressed mood, stress, anxiety, irritability, difficulty concentrating, and sleep disturbances. At low amounts, nicotine has a mild analgesic effect. Nicotine is considered a teratogen in humans. It can harm adolescent brain development, and can cause nicotine poisoning in high doses. Nicotine dependence is treatable with behavioral therapy, nicotine replacement therapy, and certain medications (e.g. bupropion and varenicline).

Methamphetamine

Methamphetamine (i.e., N-methylamphetamine) is a potent central nervous system stimulant. It is used primarily as a recreational drug, and is an aphrodisiac and euphoriant. Methamphetamine exists in three chemical forms: dextromethamphetamine (or D-methamphetamine); levomethamphetamine (or L-methamphetamine); and racemic methamphetamine. D-methamphetamine is the more psychoactive form and thus has higher potential for abuse. The racemic form also has a high potential for abuse. As such, in the United States, racemic methamphetamine and dextromethamphetamine are classified as schedule H controlled substances. L-methamphetamine has a lower potential for abuse, and has legitimate medical uses (e.g. as a nasal decongestant in the United States).

In low to moderate doses, methamphetamine can elevate mood, increase alertness, increase sexual desire, increase concentration and energy in fatigued subjects, reduce appetite, and promote weight loss. Risks of chronic and high-dose use include, for example, psychosis, skeletal muscle breakdown, seizures, bleeding in the brain, unpredictable mood swings, violent behavior, and stimulant psychosis (e.g., paranoia, hallucinations, delirium, and delusions). Such risks also include, for example, adverse changes in brain structure and function (e.g., certain reductions in grey matter volume, and adverse changes metabolic integrity markers).

At present, there are no drugs approved by the U.S. Food and Drug Administration for treating methamphetamine addiction. However, potential therapeutic approaches of interest include, for example, dopamine-partial agonists, GABA-ergic agents, and serotonergic agents.

### SUMMARY OF THE INVENTION

This invention provides a first composition of matter (“first composition”) comprising a plurality of cocaine-succinyl-BSA, wherein the average ratio of cocaine-succinyl moieties to BSA is at least 7.0.

This invention also provides a composition of matter (“first pharmaceutical composition”) comprising a plurality of cocaine-succinyl-BSA, wherein (i) the average ratio of cocaine-succinyl moieties to BSA is at least 7.0; (ii) the composition is free of EDAC; (iii) the cocaine-succinyl-BSA is adsorbed onto a suitable adjuvant; and (iv) the cocaine-succinyl-BSA so adsorbed is suspended in a suitable buffer.

This invention further provides a method (“first therapeutic method”) for treating a subject addicted to cocaine comprising administering to the subject an effective amount of the first pharmaceutical composition (i.e., a therapeutically effective amount).

This invention also provides a method (“first prophylactic method”) for inhibiting the onset of cocaine addiction in a non-addicted subject comprising administering to the subject an effective amount of the first composition (i.e., a prophylactically effect amount).

This invention provides a first synthetic method for making cocaine-succinyl-BSA, comprising the following steps:
(a) contacting cocaine with succinic anhydride in the presence of benzene under conditions permitting the formation of cocaine-succinate; and
(b) contacting the resulting cocaine-succinate with BSA under conditions permitting the formation of cocaine-succinyl-BSA, wherein (i) such conditions comprise the presence of tributylamine, dioxane, and isobutyl chloroformate, and (ii) neither step (a) nor step (b) is performed in the presence of EDAC.

This invention provides a second synthetic method for making cocaine-succinyl-BSA, comprising contacting cocaine-succinate with BSA under conditions permitting the formation of cocaine-succinyl-BSA, wherein (i) such conditions comprise the presence of tributylamine, dioxane, and isobutyl chloroformate, and (ii) the contacting is performed in the absence of EDAC.

This invention provides first and second articles of manufacture (also referred to as kits). The first article comprises (a) a composition of matter comprising a plurality of cocaine-succinyl-BSA, wherein (i) the average ratio of cocaine-succinyl moieties to BSA is at least 7.0; (ii) the composition is free of EDAC; (iii) the cocaine-succinyl-BSA is adsorbed onto a suitable adjuvant; and (iv) the cocaine-succinyl-BSA so adsorbed is suspended in a suitable buffer, and (b) a label indicating a use for the composition in treating a human subject addicted to cocaine.

The second article comprises (a) a composition of matter comprising a plurality of cocaine-succinyl-BSA, wherein (i) the average ratio of cocaine-succinyl moieties to BSA is at least 7.0; (ii) the composition is free of EDAC; (iii) the cocaine-succinyl-BSA is adsorbed onto a suitable adjuvant; and (iv) the cocaine-succinyl-BSA so adsorbed is suspended in a suitable buffer, and (b) a label indicating a use for the composition in inhibiting the onset of cocaine addiction in a non-addicted human subject.

This invention provides a second composition of matter (“second composition”) comprising a plurality of nicotine-5-succinyl-BSA, wherein the average ratio of nicotine-5-succinyl moieties to BSA is at least 7.0.

This invention also provides a composition of matter (“second pharmaceutical composition”) comprising a plurality of nicotine-5-succinyl-BSA, wherein (i) the average ratio of nicotine-5-succinyl moieties to BSA is at least 7.0; (ii) the composition is free of EDAC; (iii) the nicotine-5-succinyl-BSA is adsorbed onto a suitable adjuvant; and (iv) the nicotine-5-succinyl-BSA so adsorbed is suspended in a suitable buffer.

This invention further provides a method (“second therapeutic method”) for treating a subject addicted to nicotine comprising administering to the subject an effective amount of the second pharmaceutical composition (i.e., a therapeutically effective amount).

This invention also provides a method (“second prophylactic method”) for inhibiting the onset of nicotine addiction in a non-addicted subject comprising administering to the subject an effective amount of the second composition (i.e., a prophylactically effect amount).

This invention provides a first synthetic method for making nicotine-5-succinyl-BSA, comprising the following steps:
(a) contacting nicotine with succinic anhydride in the presence of benzene under conditions permitting the formation of nicotine-5-succinate; and
(b) contacting the resulting nicotine-5-succinate with BSA under conditions permitting the formation of nicotine-5-succinyl-BSA, wherein (i) such conditions comprise the presence of tributylamine, dioxane, and isobutyl chloroformate, and (ii) neither step (a) nor step (b) is performed in the presence of EDAC.

This invention provides a second synthetic method for making nicotine-5-succinyl-BSA, comprising contacting nicotine-5-succinate with BSA under conditions permitting the formation of nicotine-5-succinyl-BSA, wherein (i) such conditions comprise the presence of tributylamine, dioxane, and isobutyl chloroformate, and (ii) the contacting is performed in the absence of EDAC.

This invention provides third and fourth articles of manufacture (also referred to as kits). The third article comprises (a) a composition of matter comprising a plurality of nicotine-5-succinyl-BSA, wherein (i) the average ratio of nicotine-5-succinyl moieties to BSA is at least 7.0; (ii) the composition is free of EDAC; (iii) the nicotine-5-succinyl-BSA is adsorbed onto a suitable adjuvant; and (iv) the nicotine-5-succinyl-BSA so adsorbed is suspended in a suitable buffer, and (b) a label indicating a use for the composition in treating a human subject addicted to nicotine.

The fourth article comprises (a) a composition of matter comprising a plurality of nicotine-5-succinyl-BSA, wherein (i) the average ratio of nicotine-5-succinyl moieties to BSA is at least 7.0; (ii) the composition is free of EDAC; (iii) the nicotine-5-succinyl-BSA is adsorbed onto a suitable adjuvant; and (iv) the nicotine-5-succinyl-BSA so adsorbed is suspended in a suitable buffer, and (b) a label indicating a use for the composition in inhibiting the onset of nicotine addiction in a non-addicted human subject.

This invention provides a third composition of matter (“third composition”) comprising a plurality of methamphetamine-5-succinyl-BSA, wherein the average ratio of methamphetamine-5-succinyl moieties to BSA is at least 7.0.

This invention also provides a composition of matter (“third pharmaceutical composition”) comprising a plurality of methamphetamine-5-succinyl-BSA, wherein (i) the average ratio of methamphetamine-5-succinyl moieties to BSA is at least 7.0; (ii) the composition is free of EDAC; (iii) the methamphetamine-5-succinyl-BSA is adsorbed onto a suitable adjuvant; and (iv) the methamphetamine-5-succinyl-BSA so adsorbed is suspended in a suitable buffer.

This invention further provides a method (“third therapeutic method”) for treating a subject addicted to methamphetamine comprising administering to the subject an effective amount of the third pharmaceutical composition (i.e., a therapeutically effective amount).

This invention also provides a method (“third prophylactic method”) for inhibiting the onset of methamphetamine addiction in a non-addicted subject comprising administering to the subject an effective amount of the third composition (i.e., a prophylactically effect amount).

This invention provides a first synthetic method for making methamphetamine-5-succinyl-BSA, comprising the following steps:
(a) contacting methamphetamine with succinic anhydride in the presence of benzene under conditions permitting the formation of methamphetamine succinate; and
(b) contacting the resulting methamphetamine-5-succinate with BSA under conditions permitting the formation of methamphetamine-5-succinyl-BSA, wherein (i) such conditions comprise the presence of tributylamine, dioxane, and isobutyl chloroformate, and (ii) neither step (a) nor step (b) is performed in the presence of EDAC.

This invention provides a second synthetic method for making methamphetamine-5-succinyl-BSA, comprising contacting methamphetamine-5-succinate with BSA under conditions permitting the formation of methamphetamine-5-succinyl-BSA, wherein (i) such conditions comprise the presence of tributylamine, dioxane, and isobutyl chloroformate, and (ii) the contacting is performed in the absence of EDAC.

This invention provides fifth and sixth articles of manufacture (also referred to as kits). The fifth article comprises (a) a composition of matter comprising a plurality of methamphetamine-5-succinyl-BSA, wherein (i) the average ratio of methamphetamine-5-succinyl moieties to BSA is at least 7.0; (ii) the composition is free of EDAC; (iii) the methamphetamine-5-succinyl-BSA is adsorbed onto a suitable adjuvant; and (iv) the methamphetamine-5-succinyl-BSA so adsorbed is suspended in a suitable buffer, and (b) a label indicating a use for the composition in treating a human subject addicted to methamphetamine.

The sixth article comprises (a) a composition of matter comprising a plurality of methamphetamine-5-succinyl-BSA, wherein (i) the average ratio of methamphetamine-5-succinyl moieties to BSA is at least 7.0; (ii) the composition is free of EDAC; (iii) the methamphetamine-5-succinyl-BSA is adsorbed onto a suitable adjuvant; and (iv) the methamphetamine-5-succinyl-BSA so adsorbed is suspended in a suitable buffer, and (b) a label indicating a use for the composition in inhibiting the onset of methamphetamine addiction in a non-addicted human subject.

### BRIEF DESCRIPTION OF THE FIGURES

FIG. 1 This figure shows the synthesis of cocaine-5-succinyl-BSA (C-5-S-BSA). The role of the dioxane, triethylamine and isobutyl chloroformate in this reaction to is bind molecules of cocaine succinate to BSA.

FIG. 2 This figure shows a schematic of eight molecules of cocaine succinate conjugated with bovine serum albumin to form a cocaine vaccine.

FIG. 3 This figure shows the synthesis of nicotine-5-succinyl-BSA (N-5-S-BSA). In this reaction, the dioxane, triethylamine and isobutyl chloroformate bind eight molecules of nicotine-5-succinate to BSA.

FIG. 4 This figure shows a schematic of eight molecules of nicotine-5-succinate conjugated with bovine serum albumin to form a nicotine vaccine.

FIGS. 5A and 5B FIG. 5A shows possible synthetic routes (e.g., the Leuckart reaction) for making methamphetamine. FIG. 5B shows the synthesis of methamphetamine-5-succinate and methamphetamine-5-succinyl-BSA (Me-S-BSA). In this reaction, the dioxane, triethylamine and isobutyl chloroformate bind eight molecules of methamphetamine-5-succinate to BSA.

FIG. 6 This figure shows a schematic of eight molecules of methamphetamine-5-succinate conjugated with bovine serum albumin to form a methamphetamine vaccine.

### DETAILED DESCRIPTION OF THE INVENTION

Definitions

In this application, certain terms are used which shall have the meanings set forth as follows.

As used herein, “administer”, with respect to an agent, means to deliver the agent to a subject's body via any known method. Specific modes of administration include, without limitation, intravenous, intramuscular, oral, sublingual, transdermal, subcutaneous, intraperitoneal, and intrathecal administration. Preferred in this invention is intramuscular administration (e.g., injection into the deltoid muscle).

In addition, in this invention, the various agents (e.g., cocaine-succinyl-BSA, nicotine-5-succinyl-BSA, and methamphetamine-5-succinyl-BSA) used can be formulated using one or more routinely used pharmaceutically acceptable carriers appropriate for protein agents. Such carriers are well known to those skilled in the art. For example, injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone. Also included in injectable drug delivery systems are adjuvants (e.g., analgesic adjuvants; inorganic compounds (e.g., alum, aluminum hydroxide, aluminum phosphate, and calcium phosphate); mineral oil (e.g., paraffin oil); bacterial products (killed bacteria Bordetella pertussis, Mycobacterium bovis, toxoids; nonbacterial organics (e.g., squalene); plant saponins from Quillaja, soybean, polygala senega; cytokines (e.g., IL-1, IL-2, and IL-12); Freund's complete adjuvant; and Freund's incomplete adjuvant) and buffers (e.g., phosphate buffered saline) or other diluents such as sterile water.

As used herein, an “effective amount” of the first pharmaceutical composition used in the first prophylactic and therapeutic methods is an amount sufficient to deliver to the subject a prophylactic or therapeutic amount of the cocaine-succinyl-BSA (“active agent”) therein, as appropriate. In one embodiment, an effective amount of the first pharmaceutical composition contains an amount of active agent sufficient to deliver from 1 μg to 1,000 μg of active agent to the subject per dose (e.g., 1 μg, 2.5 μg, 5 μg, 7.5 μg, 10 μg, 12.5 μg, 15 μg, 20 μg, 50 μg, 100 μg, 200 μg, 300 μg, 400 μg, 500 μg, 600 μg, 700 μg, 800 μg, 900 μg, or 1,000 μg; or 1-10 μg, 10-20 μg, 20-50 μg, 50-100 μg, 100-500 μg, or 500-1,000 μg). Moreover, this dose can be administered once, or preferably a plurality of times (ideally three) over the course of prophylaxis or therapy. Similarly, as used herein, an “effective amount” of the second pharmaceutical composition used in the second prophylactic and therapeutic methods is an amount sufficient to deliver to the subject a prophylactic or therapeutic amount of the nicotine-5-succinyl-BSA (“active agent”) therein, as appropriate. In one embodiment, an effective amount of the second pharmaceutical composition contains an amount of active agent sufficient to deliver from 1 μg to 1,000 μg of active agent to the subject per dose (e.g., 1 μg, 2.5 μg, 5 μg, 7.5 μg, 10 μg, 12.5 μg, 15 μg, 20 μg, 50 μg, 100 μg, 200 μg, 300 μg, 400 μg, 500 μg, 600 μg, 700 μg, 800 μg, 900 μg, or 1,000 μg; or 1-10 μg, 10-20 μg, 20-50 μg, 50-100 μg, 100-500 μg, or 500-1,000 μg). Moreover, this dose can be administered once, or preferably a plurality of times (ideally three) over the course of prophylaxis or therapy. Similarly, as used herein, an “effective amount” of the third pharmaceutical composition used in the third prophylactic and therapeutic methods is an amount sufficient to deliver to the subject a prophylactic or therapeutic amount of the methamphetamine-5-succinyl-BSA (“active agent”) therein, as appropriate. In one embodiment, an effective amount of the third pharmaceutical composition contains an amount of active agent sufficient to deliver from 1 μg to 1,000 μg of active agent to the subject per dose (e.g., 1 μg, 2.5 μg, 5 μg, 7.5 μg, 10 μg, 12.5 μg, 15 μg, 20 μg, 50 μg, 100 μg, 200 μg, 300 μg, 400 μg, 500 μg, 600 μg, 700 μg, 800 μg, 900 μg, or 1,000 μg; or 1-10 μg, 10-20 μg, 20-50 μg, 50-100 μg, 100-500 μg, or 500-1,000 μg). Moreover, this dose can be administered once, or preferably a plurality of times (ideally three) over the course of prophylaxis or therapy.

As used herein, a composition is “free of” EDAC if, for example, it contains less than 1.0% of EDAC, less than 0.9% of EDAC, less than 0.8% of EDAC, less than 0.7% of EDAC, less than 0.6% of EDAC, less than 0.5% of EDAC, less than 0.4% of EDAC, less than 0.3% of EDAC, less than 0.2% of EDAC, less than 0.1% of EDAC, less than 0.05% of EDAC, less than 0.04% of EDAC, less than 0.03% of EDAC, less than 0.02% of EDAC, less than 0.01% of EDAC, or less than 0.001% of EDAC. If a reaction step is not “performed in the presence of EDAC”, the conditions for performing that step are “free of” EDAC. Similarly, a composition is “free of” any amidine if, for example, it contains less than 1.0% of any amidine, less than 0.9% of any amidine, less than 0.8% of any amidine, less than 0.7% of any amidine, less than 0.6% of any amidine, less than 0.5% of any amidine, less than 0.4% of any amidine, less than 0.3% of any amidine, less than 0.2% of any amidine, less than 0.1% of any amidine, less than 0.05% of any amidine, less than 0.04% of any amidine, less than 0.03% of any amidine, less than 0.02% of any amidine, less than 0.01% of any amidine, or less than 0.001% of any amidine. If a reaction step is not “performed in the presence an amidine”, the conditions for performing that step are “free of” any amidine.

As used herein, a “non-addicted” subject, with respect to cocaine, includes, without limitation, (i) a subject who has never consumed cocaine, and (ii) a subject who has consumed cocaine previously (e.g., medically or recreationally) yet is not addicted (as that term is defined herein). Similarly, as used herein, a “non-addicted” subject, with respect to nicotine, includes, without limitation, (i) a subject who has never consumed nicotine, and (ii) a subject who has consumed nicotine previously (e.g., medically or recreationally) yet is not addicted (as that term is defined herein). Similarly, as used herein, a “non-addicted” subject, with respect to methamphetamine, includes, without limitation, (i) a subject who has never consumed methamphetamine, and (ii) a subject who has consumed methamphetamine previously (e.g., medically or recreationally) yet is not addicted (as that term is defined herein).

As used herein, a “human subject” can be of any age, gender, or state of co-morbidity. In one embodiment, the subject is male, and in another, the subject is female.

As used herein, a cocaine-“addicted” subject is one who craves cocaine and, if presented with an opportunity to consume cocaine, is incapable of resisting that opportunity absent medical or therapeutic intervention. Similarly, as used herein, a nicotine-“addicted” subject is one who craves nicotine and, if presented with an opportunity to consume nicotine, is incapable of resisting that opportunity absent medical or therapeutic intervention. Similarly, as used herein, a methamphetamine-“addicted” subject is one who craves methamphetamine and, if presented with an opportunity to consume methamphetamine, is incapable of resisting that opportunity absent medical or therapeutic intervention.

As used herein, “inhibiting the onset” of cocaine addiction in a non-addicted subject includes, without limitation, reducing the likelihood of addiction by at least 10%, at least 20%, at least 30%, at least 40%, at least 50%, at least 60%, at least 70%, at least 80%, at least 90%, or at least 95%. Preferably, inhibiting the onset of cocaine addiction in a non-addicted subject means preventing such onset. Similarly, as used herein, “inhibiting the onset” of nicotine addiction in a non-addicted subject includes, without limitation, reducing the likelihood of addiction by at least 10%, at least 20%, at least 30%, at least 40%, at least 50%, at least 60%, at least 70%, at least 80%, at least 90%, or at least 95%. Preferably, inhibiting the onset of nicotine addiction in a non-addicted subject means preventing such onset. Similarly, as used herein, “inhibiting the onset” of methamphetamine addiction in a non-addicted subject includes, without limitation, reducing the likelihood of addiction by at least 10%, at least 20%, at least 30%, at least 40%, at least 50%, at least 60%, at least 70%, at least 80%, at least 90%, or at least 95%. Preferably, inhibiting the onset of methamphetamine addiction in a non-addicted subject means preventing such onset.

As used herein, the term “subject” includes, without limitation, a mammal such as a human, a non-human primate, a dog, a cat, a horse, a sheep, a goat, a cow, a rabbit, a pig, a hamster, a rat and a mouse.

As used herein, “treating” a subject addicted to cocaine and symptomatic of that addiction includes, without limitation, (i) slowing, stopping or reversing the progression of one or more symptoms of addiction, (ii) reducing or eliminating the addiction, (iii) reducing or eliminating the likelihood of the symptoms' recurrence, and/or (iv) reducing the likelihood of, or preventing, the addiction's recurrence. In the preferred embodiment, treating a subject addicted to cocaine and symptomatic of that addiction includes (i) reversing the progression of one or more of the symptoms, (ii) eliminating the addiction, (iii) preventing the symptoms' recurrence, and/or (iv) preventing the addiction's recurrence. The progress of treating a subject addicted to cocaine and symptomatic of that addiction can be measured according to a number of clinical endpoints. These endpoints include, without limitation, amelioration of one or more of the following: stroke; myocardial infarction; lung problems (for those who smoke cocaine); blood infections; sudden cardiac death; decreased ability to feel pleasure; and physical exhaustion.

Similarly, as used herein, “treating” a subject addicted to nicotine and symptomatic of that addiction includes, without limitation, (i) slowing, stopping or reversing the progression of one or more symptoms of addiction, (ii) reducing or eliminating the addiction, (iii) reducing or eliminating the likelihood of the symptoms' recurrence, and/or (iv) reducing the likelihood of, or preventing, the addiction's recurrence. In the preferred embodiment, treating a subject addicted to nicotine and symptomatic of that addiction includes (i) reversing the progression of one or more of the symptoms, (ii) eliminating the addiction, (iii) preventing the symptoms' recurrence, and/or (iv) preventing the addiction's recurrence. The progress of treating a subject addicted to nicotine and symptomatic of that addiction can be measured according to a number of clinical endpoints. These endpoints include, without limitation, amelioration of one or more of the following: depressed mood; stress; anxiety; irritability; difficulty concentrating; sleep disturbances; teratogenesis; and harm to adolescent brain development.

Similarly, as used herein, “treating” a subject addicted to methamphetamine and symptomatic of that addiction includes, without limitation, (i) slowing, stopping or reversing the progression of one or more symptoms of addiction, (ii) reducing or eliminating the addiction, (iii) reducing or eliminating the likelihood of the symptoms' recurrence, and/or (iv) reducing the likelihood of, or preventing, the addiction's recurrence. In the preferred embodiment, treating a subject addicted to methamphetamine and symptomatic of that addiction includes (i) reversing the progression of one or more of the symptoms, (ii) eliminating the addiction, (iii) preventing the symptoms' recurrence, and/or (iv) preventing the addiction's recurrence. The progress of treating a subject addicted to methamphetamine and symptomatic of that addiction can be measured according to a number of clinical endpoints. These endpoints include, without limitation, amelioration of one or more of the following: psychosis; skeletal muscle breakdown; seizures; bleeding in the brain; unpredictable mood swings; violent behavior; stimulant psychosis (e.g., paranoia, hallucinations, delirium, and delusions); and adverse changes in brain structure and function (e.g., certain reductions in grey matter volume, and adverse changes metabolic integrity markers).

Embodiments of the Invention

This application provides compositions having use as vaccines for patients dependent on cocaine, nicotine, or methamphetamine, or at risk of becoming dependent thereon. These compositions are unexpectedly superior because, among other reasons, they separately comprise cocaine-succinyl-BSA, nicotine-5-succinyl-BSA, and methamphetamine-5-succinyl-BSA having advantageously high ratios of hapten to BSA, and are free of EDAC and other harmful amidines.

Cocaine-Succinyl-BSA

Specifically, this invention provides a first composition of matter (“first composition”) comprising a plurality of cocaine-succinyl-BSA, wherein the average ratio of cocaine-succinyl moieties to BSA is at least 7.0.

In one embodiment, the first composition is free of the amidine 1-ethyl-3-(3-dimethylaminopropyl) carbodiimide (EDAC). Preferably, the first composition is also free of any amidine.

In one embodiment of the first composition, the average ratio of cocaine-succinyl moieties to BSA is selected from the group consisting of at least 7.5, at least 8.0, and at least 8.2. By way of example, in the first composition, the average ratio of cocaine-succinyl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the first composition, the average ratio of cocaine-succinyl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the first composition, the average ratio of cocaine-succinyl moieties to BSA is one of the following: 7.0-7.1; 7.1-7.2; 7.2-7.3; 7.3-7.4; 7.4-7.5; 7.5-7.6; 7.6-7.7; 7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

In a preferred embodiment, the first composition comprises a plurality of cocaine-succinyl-BSA, wherein (i) the average ratio of cocaine-succinyl moieties to BSA is at least 8.2; and (ii) the composition is free of EDAC (and is preferably free of any amidine).

This invention also provides a composition of matter (“first pharmaceutical composition”) comprising a plurality of cocaine-succinyl-BSA, wherein (i) the average ratio of cocaine-succinyl moieties to BSA is at least 7.0; (ii) the composition is free of EDAC; (iii) the cocaine-succinyl-BSA is adsorbed onto a suitable adjuvant; and (iv) the cocaine-succinyl-BSA so adsorbed is suspended in a suitable buffer. Preferably, the composition is also free of any amidine.

In one embodiment of the first pharmaceutical composition, the average ratio of cocaine-succinyl moieties to BSA is selected from the group consisting of at least 7.5, at least 8.0, and at least 8.2. By way of example, in the first pharmaceutical composition, the average ratio of cocaine-succinyl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the first pharmaceutical composition, the average ratio of cocaine-succinyl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95;

or 10.0. By way of further example, in the first pharmaceutical composition, the average ratio of cocaine-succinyl moieties to BSA is one of the following: 7.0-7.1; 7.1-7.2; 7.2-7.3; 7.3-7.4; 7.4-7.5; 7.5-7.6; 7.6-7.7; 7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

Adjuvants suitable for use in the first pharmaceutical composition are known. In a preferred embodiment, the suitable adjuvant is aluminum hydroxide gel. Similarly, buffers suitable for use in the first pharmaceutical composition are known. In a preferred embodiment, the suitable buffer is phosphate buffered saline.

In a preferred embodiment, the first pharmaceutical composition comprises a plurality of cocaine-succinyl-BSA, wherein (i) the average ratio of cocaine-succinyl moieties to BSA is at least 8.2; (ii) the composition is free of EDAC (and preferably is free of any amidine); (iii) the cocaine-succinyl-BSA is adsorbed onto aluminum hydroxide gel; and (iv) the cocaine-succinyl-BSA so adsorbed is suspended in phosphate buffered saline.

This invention further provides a method (“first therapeutic method”) for treating a subject addicted to cocaine comprising administering to the subject an effective amount of the first pharmaceutical composition (i.e., a therapeutically effective amount). Preferably, the subject is human.

In the first therapeutic method, the first pharmaceutical composition can be administered according to any appropriate dosing regimen. In one embodiment, the first pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more “boosters” at suitable intervals (e.g., every two or three years).

In a preferred embodiment of the first therapeutic method, the method comprises injecting the subject with an effective amount of the first pharmaceutical composition, wherein the composition is administered via three doses at 30-day intervals. Preferably, the subject is human.

This invention also provides a method (“first prophylactic method”) for inhibiting the onset of cocaine addiction in a non-addicted subject comprising administering to the subject an effective amount of the first composition (i.e., a prophylactically effect amount). Preferably, the subject is human.

In the first prophylactic method, the first pharmaceutical composition can be administered according to any appropriate dosing regimen. In one embodiment, the first pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more “boosters” at suitable intervals (e.g., every two or three years).

In a preferred embodiment of the first prophylactic method, the method comprises injecting the human subject with an effective amount of the first prophylactic composition, wherein the composition is administered via three doses at 30-day intervals.

The first composition (as opposed to the first pharmaceutical composition) is also envisioned, mutatis mutandis, for use in the present therapeutic and prophylactic methods.

This invention provides a first synthetic method for making cocaine-succinyl-BSA, comprising the following steps:
(a) contacting cocaine with succinic anhydride in the presence of benzene under conditions permitting the formation of cocaine-succinate; and
(b) contacting the resulting cocaine-succinate with BSA under conditions permitting the formation of cocaine-succinyl-BSA, wherein (i) such conditions comprise the presence of tributylamine, dioxane, and isobutyl chloroformate, and (ii) neither step (a) nor step (b) is performed in the presence of EDAC.

In a preferred embodiment of the first synthetic method, neither step (a) nor step (b) is performed in the presence of an amidine.

This invention provides a second synthetic method for making cocaine-succinyl-BSA, comprising contacting cocaine-succinate with BSA under conditions permitting the formation of cocaine-succinyl-BSA, wherein (i) such conditions comprise the presence of tributylamine, dioxane, and isobutyl chloroformate, and (ii) the contacting is performed in the absence of EDAC.

In a preferred embodiment of the second synthetic method, the contacting is performed in the absence of any amidine.

This invention provides first and second articles of manufacture (also referred to as kits). The first article comprises (a) a composition of matter comprising a plurality of cocaine-succinyl-BSA, wherein (i) the average ratio of cocaine-succinyl moieties to BSA is at least 7.0; (ii) the composition is free of EDAC; (iii) the cocaine-succinyl-BSA is adsorbed onto a suitable adjuvant; and (iv) the cocaine-succinyl-BSA so adsorbed is suspended in a suitable buffer, and (b) a label indicating a use for the composition in treating a human subject addicted to cocaine.

In a preferred embodiment, the first article comprises (a) a composition of matter comprising a plurality of cocaine-succinyl-BSA, wherein (i) the average ratio of cocaine-succinyl moieties to BSA is at least 8.2; (ii) the composition is free of EDAC; (iii) the cocaine-succinyl-BSA is adsorbed onto aluminum hydroxide gel; and (iv) the cocaine-succinyl-BSA so adsorbed is suspended in phosphate buffered saline, and (b) a label indicating a use for the composition in treating a human subject addicted to cocaine.

The second article comprises (a) a composition of matter comprising a plurality of cocaine-succinyl-BSA, wherein (i) the average ratio of cocaine-succinyl moieties to BSA is at least 7.0; (ii) the composition is free of EDAC; (iii) the cocaine-succinyl-BSA is adsorbed onto a suitable adjuvant; and (iv) the cocaine-succinyl-BSA so adsorbed is suspended in a suitable buffer, and (b) a label indicating a use for the composition in inhibiting the onset of cocaine addiction in a non-addicted human subject.

In a preferred embodiment, the second article comprises (a) a composition of matter comprising a plurality of cocaine-succinyl-BSA, wherein (i) the average ratio of cocaine-succinyl moieties to BSA is at least 8.2; (ii) the composition is free of EDAC; (iii) the cocaine-succinyl-BSA is adsorbed onto aluminum hydroxide gel; and (iv) the cocaine-succinyl-BSA so adsorbed is suspended in phosphate buffered saline, and (b) a label indicating a use for the composition in inhibiting the onset of cocaine addiction in a non-addicted human subject.

Preferably, in the first and second articles, the composition is free of any amidine.

Nicotine-5-Succinyl-BSA

This invention provides a composition of matter (“second composition”) comprising a plurality of nicotine-5-succinyl-BSA, wherein the average ratio of nicotine-5-succinyl moieties to BSA is at least 7.0.

In one embodiment, the second composition is free of the amidine 1-ethyl-3-(3-dimethylaminopropyl) carbodiimide (EDAC). Preferably, the second composition is also free of any amidine.

In one embodiment of the second composition, the average ratio of nicotine-5-succinyl moieties to BSA is selected from the group consisting of at least 7.5, at least 8.0, and at least 8.2. By way of example, in the second composition, the average ratio of nicotine-5-succinyl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the second composition, the average ratio of nicotine-5-succinyl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the second composition, the average ratio of nicotine-5-succinyl moieties to BSA is one of the following: 7.0-7.1; 7.1-7.2; 7.2-7.3; 7.3-7.4; 7.4-7.5; 7.5-7.6; 7.6-7.7;7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

In a preferred embodiment, the second composition comprises a plurality of nicotine-5-succinyl-BSA, wherein (i) the average ratio of nicotine-5-succinyl moieties to BSA is at least 8.2; and (ii) the composition is free of EDAC (and is preferably free of any amidine).

This invention also provides a composition of matter (“second pharmaceutical composition”) comprising a plurality of nicotine-5-succinyl-BSA, wherein (i) the average ratio of nicotine-5-succinyl moieties to BSA is at least 7.0; (ii) the composition is free of EDAC; (iii) the nicotine-5-succinyl-BSA is adsorbed onto a suitable adjuvant; and (iv) the nicotine-5-succinyl-BSA so adsorbed is suspended in a suitable buffer. Preferably, the composition is also free of any amidine.

In one embodiment of the second pharmaceutical composition, the average ratio of nicotine-5-succinyl moieties to BSA is selected from the group consisting of at least 7.5, at least 8.0, and at least 8.2. By way of example, in the second pharmaceutical composition, the average ratio of nicotine-5-succinyl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the second pharmaceutical composition, the average ratio of nicotine-5-succinyl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the second pharmaceutical composition, the average ratio of nicotine-5-succinyl moieties to BSA is one of the following: 7.0-7.1; 7.1-7.2; 7.2-7.3; 7.3-7.4; 7.4-7.5; 7.5-7.6; 7.6-7.7; 7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

Adjuvants suitable for use in the second pharmaceutical composition are known. In a preferred embodiment, the suitable adjuvant is aluminum hydroxide gel. Similarly, buffers suitable for use in the second pharmaceutical composition are known. In a preferred embodiment, the suitable buffer is phosphate buffered saline.

In a preferred embodiment, the second pharmaceutical composition comprises a plurality of nicotine-5-succinyl-BSA, wherein (i) the average ratio of nicotine-5-succinyl moieties to BSA is at least 8.2; (ii) the composition is free of EDAC (and preferably is free of any amidine); (iii) the nicotine-5-succinyl-BSA is adsorbed onto aluminum hydroxide gel; and (iv) the nicotine-5-succinyl-BSA so adsorbed is suspended in phosphate buffered saline.

This invention further provides a method (“second therapeutic method”) for treating a subject addicted to nicotine comprising administering to the subject an effective amount of the second pharmaceutical composition (i.e., a therapeutically effective amount). Preferably, the subject is human.

In the second therapeutic method, the second pharmaceutical composition can be administered according to any appropriate dosing regimen. In one embodiment, the second pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more boosters at suitable intervals (e.g., every two or three years).

In a preferred embodiment of the second therapeutic method, the method comprises injecting the subject with an effective amount of the second pharmaceutical composition, wherein the composition is administered via three doses at 30-day intervals. Preferably, the subject is human.

This invention also provides a method (“second prophylactic method”) for inhibiting the onset of nicotine addiction in a non-addicted subject comprising administering to the subject an effective amount of the second composition (i.e., a prophylactically effect amount). Preferably, the subject is human.

In the second prophylactic method, the second pharmaceutical composition can be administered according to any appropriate dosing regimen. In one embodiment, the second pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more “boosters” at suitable intervals (e.g., every two or three years).

In a preferred embodiment of the second prophylactic method, the method comprises injecting the human subject with an effective amount of the second prophylactic composition, wherein the composition is administered via three doses at 30-day intervals.

The second composition (as opposed to the second pharmaceutical composition) is also envisioned, mutatis mutandis, for use in the present therapeutic and prophylactic methods.

This invention provides a first synthetic method for making nicotine-5-succinyl-BSA, comprising the following steps:
(a) contacting nicotine with succinic anhydride in the presence of benzene under conditions permitting the formation of nicotine-5-succinate; and
(b) contacting the resulting nicotine-5-succinate with BSA under conditions permitting the formation of nicotine-5-succinyl-BSA, wherein (i) such conditions comprise the presence of tributylamine, dioxane, and isobutyl chloroformate, and (ii) neither step (a) nor step (b) is performed in the presence of EDAC.

In a preferred embodiment of the first synthetic method, neither step (a) nor step (b) is performed in the presence of an amidine.

This invention provides a second synthetic method for making nicotine-5-succinyl-BSA, comprising contacting nicotine-5-succinate with BSA under conditions permitting the formation of nicotine-5-succinyl-BSA, wherein (i) such conditions comprise the presence of tributylamine, dioxane, and isobutyl chloroformate, and (ii) the contacting is performed in the absence of EDAC.

In a preferred embodiment of the second synthetic method, the contacting is performed in the absence of any amidine.

This invention provides third and fourth articles of manufacture (also referred to as kits). The third article comprises (a) a composition of matter comprising a plurality of nicotine-5-succinyl-BSA, wherein (i) the average ratio of nicotine-5-succinyl moieties to BSA is at least 7.0; (ii) the composition is free of EDAC; (iii) the nicotine-5-succinyl-BSA is adsorbed onto a suitable adjuvant; and (iv) the nicotine-5-succinyl-BSA so adsorbed is suspended in a suitable buffer, and (b) a label indicating a use for the composition in treating a human subject addicted to nicotine.

In a preferred embodiment, the third article comprises (a) a composition of matter comprising a plurality of nicotine-5-succinyl-BSA, wherein (i) the average ratio of nicotine-5-succinyl moieties to BSA is at least 8.2; (ii) the composition is free of EDAC; (iii) the nicotine-5-succinyl-BSA is adsorbed onto aluminum hydroxide gel; and (iv) the nicotine-5-succinyl-BSA so adsorbed is suspended in phosphate buffered saline, and (b) a label indicating a use for the composition in treating a human subject addicted to nicotine.

The fourth article comprises (a) a composition of matter comprising a plurality of nicotine-5-succinyl-BSA, wherein (i) the average ratio of nicotine-5-succinyl moieties to BSA is at least 7.0; (ii) the composition is free of EDAC; (iii) the nicotine-5-succinyl-BSA is adsorbed onto a suitable adjuvant; and (iv) the nicotine-5-succinyl-BSA so adsorbed is suspended in a suitable buffer, and (b) a label indicating a use for the composition in inhibiting the onset of nicotine addiction in a non-addicted human subject.

In a preferred embodiment, the fourth article comprises (a) a composition of matter comprising a plurality of nicotine-5-succinyl-BSA, wherein (i) the average ratio of nicotine-5-succinyl moieties to BSA is at least 8.2; (ii) the composition is free of EDAC; (iii) the nicotine-5-succinyl-BSA is adsorbed onto aluminum hydroxide gel; and (iv) the nicotine-5-succinyl-BSA so adsorbed is suspended in phosphate buffered saline, and (b) a label indicating a use for the composition in inhibiting the onset of nicotine addiction in a non-addicted human subject.

Preferably, in the third and fourth articles, the composition is free of any amidine.

Methamphetamine-5-Succinyl-BSA

This invention provides a composition of matter (“third composition”) comprising a plurality of methamphetamine-5-succinyl-BSA, wherein the average ratio of methamphetamine-5-succinyl moieties to BSA is at least 7.0.

The methamphetamine-5-succinyl-BSA in this third composition can be, for example, D-methamphetamine-5-succinyl-BSA, L-methamphetamine-5-succinyl-BSA, or racemic methamphetamine-5-succinyl-BSA. So, in the preferred embodiment of this third composition, each BSA has bound to it eight molecules of D-methamphetamine-5-succinate. In another embodiment, each BSA has bound to it eight molecules of L-methamphetamine-5-succinate. In a further embodiment, some BSAs have bound to them eight molecules of D-methamphetamine-5-succinate, and other BSAs have bound to them eight molecules of L-methamphetamine-5-succinate. In yet a further embodiment, each BSA has bound to it (i) eight molecules of D-methamphetamine-5-succinate; (ii) eight molecules of L-methamphetamine-5-succinate; or (iii) eight molecules of methamphetamine-5-succinate having a D:L ratio of 1:7, 2:6, 3:5, 4:4, 5:3, 6:2, or 7:1.

In one embodiment, the third composition is free of the amidine 1-ethyl-3-(3-dimethylaminopropyl) carbodiimide (EDAC). Preferably, the third composition is also free of any amidine.

In one embodiment of the third composition, the average ratio of methamphetamine succinyl moieties to BSA is selected from the group consisting of at least 7.5, at least 8.0, and at least 8.2. By way of example, in the third composition, the average ratio of methamphetamine-5-succinyl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the third composition, the average ratio of methamphetamine-5-succinyl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the third composition, the average ratio of methamphetamine-5-succinyl moieties to BSA is one of the following: 7.0-7.1; 7.1-7.2; 7.2-7.3; 7.3-7.4; 7.4-7.5; 7.5-7.6; 7.6-7.7; 7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

In a preferred embodiment, the third composition comprises a plurality of methamphetamine-5-succinyl-BSA, wherein (i) the average ratio of methamphetamine-5-succinyl moieties to BSA is at least 8.2; and (ii) the composition is free of EDAC (and is preferably free of any amidine).

This invention also provides a composition of matter (“third pharmaceutical composition”) comprising a plurality of methamphetamine-5-succinyl-BSA, wherein (i) the average ratio of methamphetamine-5-succinyl moieties to BSA is at least 7.0; (ii) the composition is free of EDAC; (iii) the methamphetamine-5-succinyl-BSA is adsorbed onto a suitable adjuvant; and (iv) the methamphetamine-5-succinyl-BSA so adsorbed is suspended in a suitable buffer. Preferably, the composition is also free of any amidine.

In one embodiment of the third pharmaceutical composition, the average ratio of methamphetamine-5-succinyl moieties to BSA is selected from the group consisting of at least 7.5, at least 8.0, and at least 8.2. By way of example, in the third pharmaceutical composition, the average ratio of methamphetamine-5-succinyl moieties to BSA is at least one of the following: 7.0; 7.1; 7.2; 7.3; 7.4; 7.5; 7.6; 7.7; 7.8; 7.9; 8.0; 8.05; 8.1; 8.15; 8.2; 8.25; 8.3; 8.35; 8.4; 8.45; 8.5; 8.6; 8.7; 8.8; 8.9; 9.0; 9.1; 9.2; 9.3; 9.4; 9.5; 9.6; 9.7; 9.8; 9.9; or 10.0.

By way of example, in the third pharmaceutical composition, the average ratio of methamphetamine-5-succinyl moieties to BSA is at least one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1; 9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the third pharmaceutical composition, the average ratio of methamphetamine-5-succinyl moieties to BSA is one of the following: 7.0; 7.05; 7.1; 7.15; 7.2; 7.25; 7.3; 7.35; 7.4; 7.45; 7.5; 7.55; 7.6; 7.65; 7.7; 7.75; 7.8; 7.85; 7.9; 7.95; 8.0; 8.05; 8.1; 8.15; 8.2; 8.25; 8.3; 8.35; 8.4; 8.45; 8.5; 8.55; 8.6; 8.65; 8.7; 8.75; 8.8; 8.85; 8.9; 8.95; 9.0; 9.05; 9.1;

9.15; 9.2; 9.25; 9.3; 9.35; 9.4; 9.45; 9.5; 9.55; 9.6; 9.65; 9.7; 9.75; 9.8; 9.85; 9.9; 9.95; or 10.0. By way of further example, in the third pharmaceutical composition, the average ratio of methamphetamine-5-succinyl moieties to BSA is one of the following: 7.0-7.1; 7.1-7.2; 7.2-7.3; 7.3-7.4; 7.4-7.5; 7.5-7.6; 7.6-7.7; 7.7-7.8; 7.8-7.9; 7.9-8.0; 8.0-8.1; 8.1-8.2; 8.2-8.3; 8.3-8.4; 8.4-8.5; 8.5-8.6; 8.6-8.7; 8.7-8.8; 8.8-8.9; 8.9-9.0; 9.0-9.1; 9.1-9.2; 9.2-9.3; 9.3-9.4; 9.4-9.5; 9.5-9.6; 9.6-9.7; 9.7-9.8; 9.8-9.9; or 9.9-10.0; 7.0-7.2; 7.2-7.4; 7.4-7.6; 7.6-7.8; 7.8-8.0; 8.0-8.2; 8.2-8.4; 8.4-8.6; 8.6-8.8; 8.8-9.0; 9.0-9.2; 9.2-9.4; 9.4-9.6; 9.6-9.8; or 9.8-10.0.

Adjuvants suitable for use in the third pharmaceutical composition are known. In a preferred embodiment, the suitable adjuvant is aluminum hydroxide gel. Similarly, buffers suitable for use in the third pharmaceutical composition are known. In a preferred embodiment, the suitable buffer is phosphate buffered saline.

In a preferred embodiment, the third pharmaceutical composition comprises a plurality of methamphetamine-5-succinyl-BSA, wherein (i) the average ratio of methamphetamine-5-succinyl moieties to BSA is at least 8.2; (ii) the composition is free of EDAC (and preferably is free of any amidine); (iii) the methamphetamine-5-succinyl-BSA is adsorbed onto aluminum hydroxide gel; and (iv) the methamphetamine succinyl-BSA so adsorbed is suspended in phosphate buffered saline.

This invention further provides a method (“third therapeutic method”) for treating a subject addicted to methamphetamine comprising administering to the subject an effective amount of the third pharmaceutical composition (i.e., a therapeutically effective amount). Preferably, the subject is human.

In the third therapeutic method, the third pharmaceutical composition can be administered according to any appropriate dosing regimen. In one embodiment, the third pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more boosters at suitable intervals (e.g., every two or three years).

In a preferred embodiment of the third therapeutic method, the method comprises injecting the subject with an effective amount of the third pharmaceutical composition, wherein the composition is administered via three doses at 30-day intervals. Preferably, the subject is human.

This invention also provides a method (“third prophylactic method”) for inhibiting the onset of methamphetamine addiction in a non-addicted subject comprising administering to the subject an effective amount of the third composition (i.e., a prophylactically effect amount). Preferably, the subject is human.

In the third prophylactic method, the third pharmaceutical composition can be administered according to any appropriate dosing regimen. In one embodiment, the third pharmaceutical composition is administered via a single dose. In a preferred embodiment, the composition is administered via a plurality of doses, such as two doses 30 days apart. In a further preferred embodiment, the composition is administered via three doses at 30-day intervals (i.e., at day 0, day 30 and day 60). In yet another embodiment, this administration is followed with one or more “boosters” at suitable intervals (e.g., every two or three years).

In a preferred embodiment of the third prophylactic method, the method comprises injecting the human subject with an effective amount of the third prophylactic composition, wherein the composition is administered via three doses at 30-day intervals.

The third composition (as opposed to the third pharmaceutical composition) is also envisioned, mutatis mutandis, for use in the present therapeutic and prophylactic methods.

This invention provides a first synthetic method for making methamphetamine-5-succinyl-BSA, comprising the following steps:
(a) contacting methamphetamine with succinic anhydride in the presence of benzene under conditions permitting the formation of methamphetamine-5-succinate; and
(b) contacting the resulting methamphetamine-5-succinate with BSA under conditions permitting the formation of methamphetamine-5-succinyl-BSA, wherein (i) such conditions comprise the presence of tributylamine, dioxane, and isobutyl chloroformate, and (ii) neither step (a) nor step (b) is performed in the presence of EDAC.

In a preferred embodiment of the first synthetic method, neither step (a) nor step (b) is performed in the presence of an amidine.

This invention provides a second synthetic method for making methamphetamine-5-succinyl-BSA, comprising contacting methamphetamine-5-succinate with BSA under conditions permitting the formation of methamphetamine-5-succinyl-BSA, wherein (i) such conditions comprise the presence of tributylamine, dioxane, and isobutyl chloroformate, and (ii) the contacting is performed in the absence of EDAC.

In a preferred embodiment of the second synthetic method, the contacting is performed in the absence of any amidine.

This invention provides fifth and sixth articles of manufacture (also referred to as kits). The fifth article comprises (a) a composition of matter comprising a plurality of methamphetamine-5-succinyl-BSA, wherein (i) the average ratio of methamphetamine-5-succinyl moieties to BSA is at least 7.0; (ii) the composition is free of EDAC; (iii) the methamphetamine-5-succinyl-BSA is adsorbed onto a suitable adjuvant; and (iv) the methamphetamine-5-succinyl-BSA so adsorbed is suspended in a suitable buffer, and (b) a label indicating a use for the composition in treating a human subject addicted to methamphetamine.

In a preferred embodiment, the fifth article comprises (a) a composition of matter comprising a plurality of methamphetamine-5-succinyl-BSA, wherein (i) the average ratio of methamphetamine-5-succinyl moieties to BSA is at least 8.2; (ii) the composition is free of EDAC; (iii) the methamphetamine-5-succinyl-BSA is adsorbed onto aluminum hydroxide gel; and (iv) the methamphetamine-5-succinyl-BSA so adsorbed is suspended in phosphate buffered saline, and (b) a label indicating a use for the composition in treating a human subject addicted to methamphetamine.

The sixth article comprises (a) a composition of matter comprising a plurality of methamphetamine-5-succinyl-BSA, wherein (i) the average ratio of methamphetamine-5-succinyl moieties to BSA is at least 7.0; (ii) the composition is free of EDAC; (iii) the methamphetamine-5-succinyl-BSA is adsorbed onto a suitable adjuvant; and (iv) the methamphetamine-5-succinyl-BSA so adsorbed is suspended in a suitable buffer, and (b) a label indicating a use for the composition in inhibiting the onset of methamphetamine addiction in a non-addicted human subject.

In a preferred embodiment, the sixth article comprises (a) a composition of matter comprising a plurality of methamphetamine-5-succinyl-BSA, wherein (i) the average ratio of methamphetamine-5-succinyl moieties to BSA is at least 8.2; (ii) the composition is free of EDAC; (iii) the methamphetamine-5-succinyl-BSA is adsorbed onto aluminum hydroxide gel; and (iv) the methamphetamine-5-succinyl-BSA so adsorbed is suspended in phosphate buffered saline, and (b) a label indicating a use for the composition in inhibiting the onset of methamphetamine addiction in a non-addicted human subject.

Preferably, in the fifth and sixth articles, the composition is free of any amidine.

This invention will be better understood by reference to the examples which follow, but those skilled in the art will readily appreciate that the specific examples detailed are only illustrative of the invention as described more fully in the claims which follow thereafter.

### EXAMPLES

### Example 1

### Production of Cocaine-5-Succinate (C-5-S)

The reaction of cocaine with succinic anhydride in benzene leads to the selective succinylation of the 5-hydroxyl cocaine group.

Specifically, 0.035 mol cocaine with 0.1 mol succinic anhydride in benzene is poured into a 1-liter double-spouted balloon, to which is added 0.1 mol succinic anhydride again under refrigerant after two to three hours of boiling.

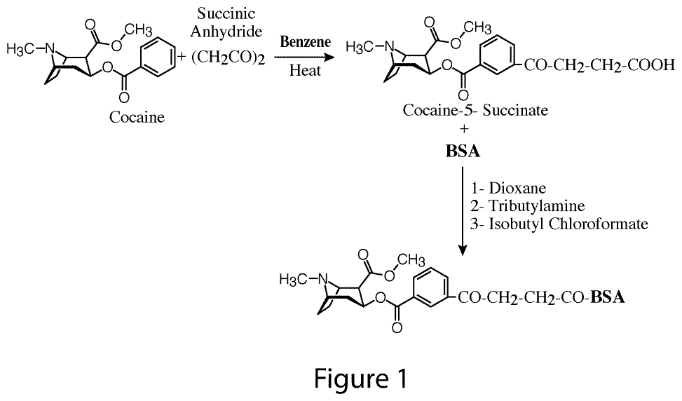

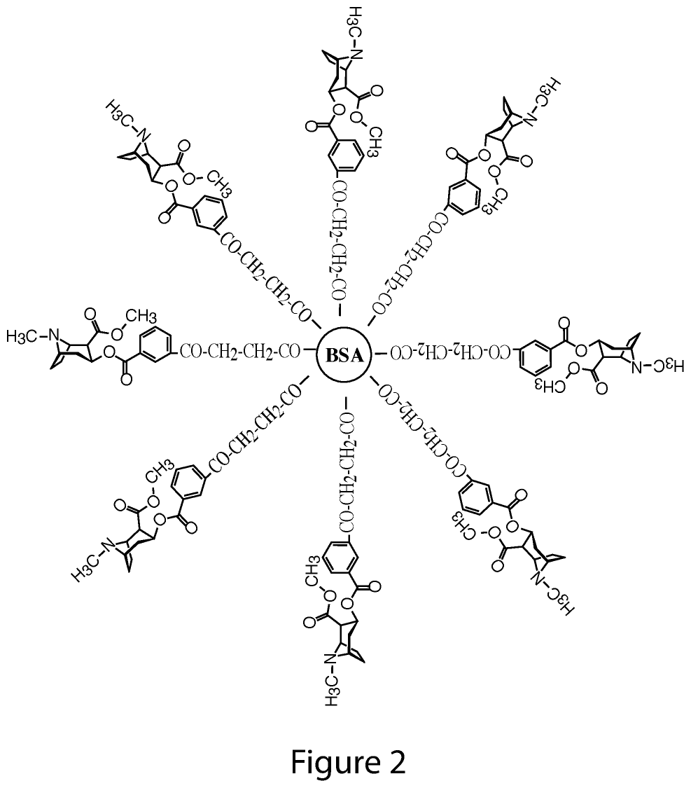

The reaction mixture is cooled to room temperature, benzene is slowly removed, and the residual benzene evaporated with the help of nitrogen gas flow. After the mixture inside the balloon is completely benzene-free, the residue is mixed in 1,000 ml of distilled water, its pH is brought to 5 with HCl, it is filtered with a Buchner funnel with succinic anhydride, and is filtered again. The resulting paste is cocaine-5-succinate. The pH of the filter solution is then raised to 8.5 and the solution is filtered again to remove cocaine from the reaction. The pH of the cocaine-5-succinate (C-5-S) filtrate solution is then adjusted to 7 and refrigerated overnight to crystallize the C-5-S. The crystals are separated by Buchner funnel filtration in the presence of anhydrous CaCl₂ in vacuo at 60° C. in a lactic desiccator. Crystallized cocaine-5-succinate is stored in brown closed vials. See FIG. 1.

### Example 2

### Production of Cocaine-5-Succinyl-BSA (C-5-S-BSA)

Tri-N-butyl amine, dioxane, isobutyl chloroformate and sodium hydroxide can be purchased from Merck (Germany). In a three-liter one-way balloon, 2.15 mM cocaine-5-succinate and 2.53 mM Trim-N-butyl amine are mixed in 21.5 mL dioxane, After the mixture is brought to 10° C., 2.55 mM isobutyl chloroformate is added. The reaction is continued at room temperature for 60 minutes. The mixture is then immediately dissolved in a 5° C. cold solution of 0.051 mmol BSA (dissolved in 186 mL 1:1 water-dioxane; spectral grade) and 3.58 mL normal NaOH while mixing well. Following the addition of the solution, the color becomes slightly opaque and gas bubbles appear. The pH of the solution is then adjusted to 7.5 with normal NaOH. After one hour with NaOH, the pH of the solution is adjusted to 7, and mixing continues at 5° C. for more 2 hours. The product is poured into a dialysis bag with a cuff of 30,000 Daltons and dialyzed overnight under low-flow urban (tap) water. Then, in a beaker, the pH is adjusted to 4.5 with HCl. Cocaine-5-succinyl-BSA begins to precipitate. The precipitate is kept inside the beaker at 4° C. for one night, and then separated via centrifuge the next day. The precipitate of cocaine-5-succinyl-BSA is suspended in 100 ml of distilled water and brought to pH 7 with 0.1 NaHCO3 solution. Then, the product is dialyzed under tap water for 4 hours. The product is dissolved in pyrogen-free distilled water, and filtration and sterilization are continued. Ten doses may be used for quality control, waste management, and expiration. See FIG. 1.

### Example 3

### Cocaine Vaccine Formulation (C-5-S-BSA)

| | The role | Formulation |
|---|---|---|
| | of each | values of one |
| Components Formulation | component | dose per ml |
| Antigenic protein | Immunogen | 50 ug |
| (C-5-S-BSA) | | |
| Aluminum hydroxide | Adjuvant | 0.5 mg |
| Sodium chloride | Buffer | 8 mg |
| Anhydrous disodium | Buffer | 1.12 mg |
| hydrogen phosphate | | |
| Sodium dihydrogen | Buffer | 1.1 mg |
| phosphate monohydrate | | |
| Injectable distilled water Qs | Solvent | 1 ml |

### Example 4

### Production of Nicotine-5-Succinyl-BSA (N-5-S-BSA)

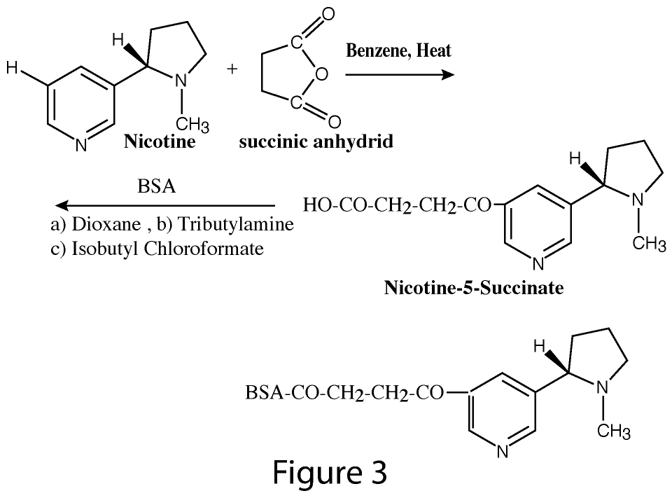

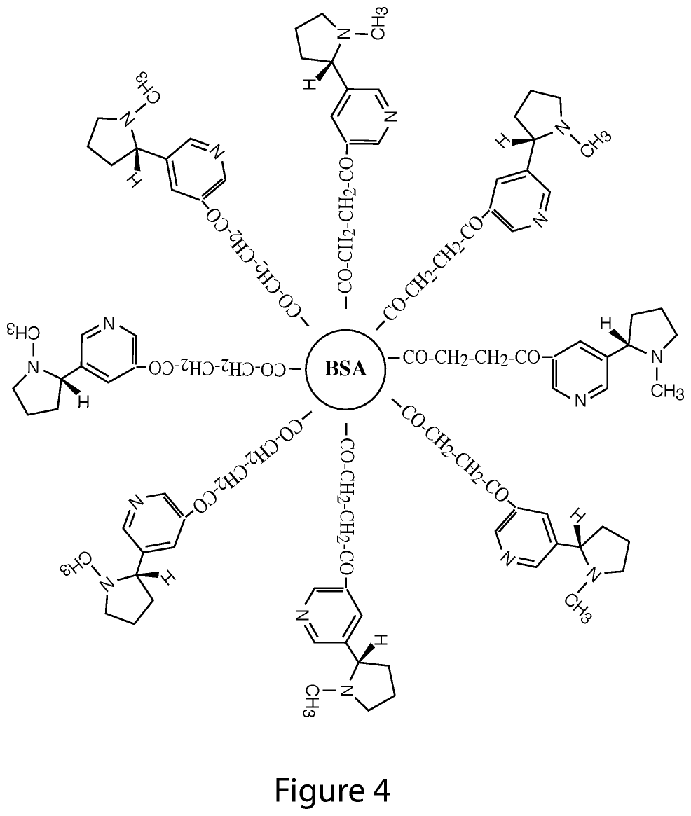

The reaction of nicotine with succinic anhydride in benzene yields nicotine-5-succinate. Nicotine-5-succinate is reacted with BSA (i.e., BSA-(CH2)4-NH2), tributyl amine, dioxane, and isobutyl chloroformate to yield nicotine-5-succinyl-BSA (also referred to as BSA-nicotine-6-succinate). See FIG. 3. The reaction conditions used in the production of cocaine-5-succinyl-BSA in Examples 1 and 2 above can also be used to produce nicotine-5-succinyl-BSA.

### Example 5

### Nicotine Vaccine Formulation (N-5-S-BSA)

| | The role | Formulation |
|---|---|---|
| | of each | values of one |
| Components Formulation | component | dose per ml |
| Antigenic protein | Immunogen | 50 ug |
| (N-5-S-BSA) | | |
| Aluminum hydroxide | Adjuvant | 0.5 mg |
| Sodium chloride | Buffer | 8 mg |
| Anhydrous disodium | Buffer | 1.12 mg |
| hydrogen phosphate | | |
| Sodium dihydrogen | Buffer | 1.1 mg |
| phosphate monohydrate | | |
| Injectable distilled water Qs | Solvent | 1 ml |

### Example 6

### Production of Methamphetamine-5-Succinyl-BSA (Me-5-S-BSA)

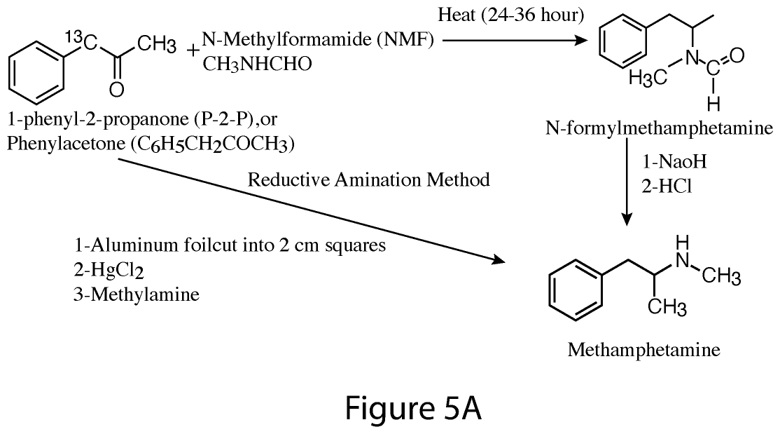

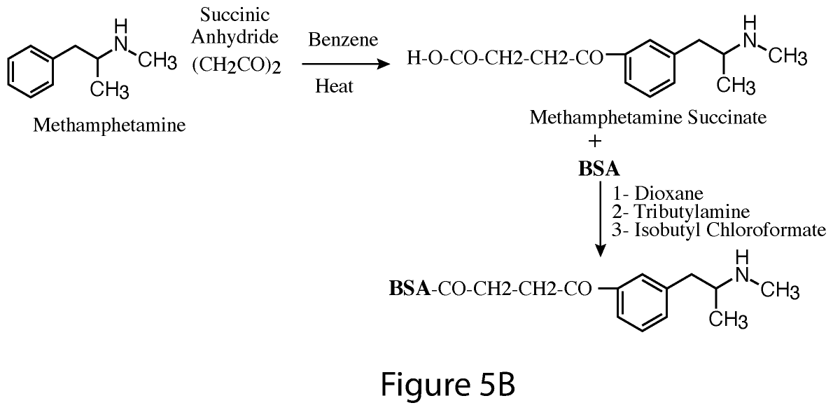

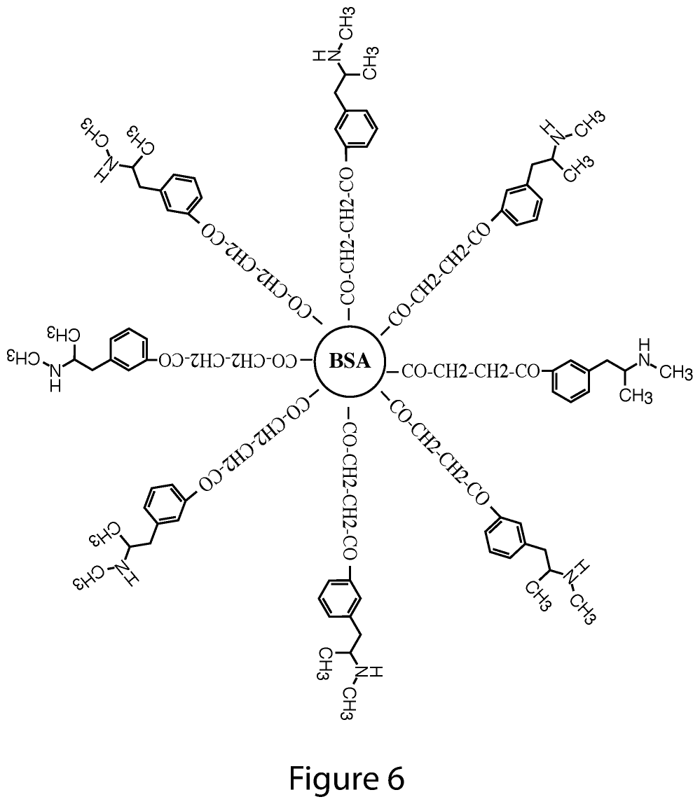

The reaction of methamphetamine (preferably D-methamphetamine) with succinic anhydride in benzene yields methamphetamine-5-succinate (preferably D-methamphetamine-5-succinate). Methamphetamine-5-succinate is reacted with BSA (i.e., BSA-(CH₂)₄-NH₂), tributyl amine, dioxane, and isobutyl chloroformate to yield methamphetamine-5-succinyl-BSA (also referred to as BSA-methamphetamine-5-succinate). Preferably, the methamphetamine-5-succinyl-BSA is D-methamphetamine-5-succinyl-BSA. See FIG. 5B. Methamphetamine-5-succinyl-BSA can be produced using L-methamphetamine or racemic methamphetamine as well, although D-methamphetamine is preferred. The reaction conditions used in the production of cocaine-5-succinyl-BSA in Examples 1 and 2 above can also be used to produce methamphetamine-5-succinyl-BSA.

### Example 7

### Methamphetamine Vaccine Formulation (Me-5-S-BSA)

The following table sets forth the contents of a 1 ml dosage form (vial) of the subject methamphetamine vaccine (i.e., methamphetamine-5-succinyl-BSA). Preferably, the methamphetamine-5-succinyl-BSA in this dosage form is D-methamphetamine-5-succinyl-BSA.

| | The role | Formulation |
|---|---|---|
| | of each | values of one |
| Components Formulation | component | dose per ml |
| Antigenic protein | Immunogen | 50 ug |
| (Me-5-S-BSA) | | |
| Aluminum hydroxide | Adjuvant | 0.5 mg |
| Sodium chloride | Buffer | 8 mg |
| Anhydrous disodium | Buffer | 1.12 mg |
| hydrogen phosphate | | |
| Sodium dihydrogen | Buffer | 1.1 mg |
| phosphate monohydrate | | |
| Injectable distilled water Qs | Solvent | 1 ml |

### REFERENCES

1. A. Akbarzadeh, et al., Asian J. of Biochemistry, 2(1):58-65 (2007).
2. A. Akbarzadeh, et al., J. of Pharm. And Toxicol., 4(1):3035 (2009).
3. A. Akbarzadeh, et al., Biotechnology and Applied Biochem., (Dec. 23, 2010).
4. F. Li, et al., Org. Biomol. Chem. 2014 Oct. 7; 12(37): 7211-7232.
5. M. Pravetoni, et al., Biochem. Pharmacol. 2012 Feb. 15; 83(4):543-50.
6. M. Pravetoni, et al., J. Pharmacol. Exp. Ther. 2012 April; 341(1):225-32.
7. Y. Hu, et al., Hum. Vaccin. Immunother. 2014; 10(1):64-72.

1. A composition of matter comprising a plurality of cocaine-succinyl-bovine serum albumin (BSA), wherein the average ratio of cocaine-succinyl moieties to BSA is at least 7.0.

2. The composition of claim 1, wherein the composition is free of 1-ethyl-3-(3-dimethylaminopropyl) carbodiimide (EDAC) and free of any amidine.

3. (canceled)

4. The composition of claim 1, wherein the average ratio of cocaine-succinyl moieties to BSA is selected from the group consisting of at least 7.5, at least 8.0, and at least 8.2.

5. A composition of matter comprising a plurality of cocaine-succinyl-BSA, wherein (i) the average ratio of cocaine-succinyl moieties to BSA is at least 8.2; and (ii) the composition is free of EDAC and free of any amidine.

6. (canceled)

7. A composition of matter comprising a plurality of cocaine-succinyl-BSA, wherein (i) the average ratio of cocaine-succinyl moieties to BSA is at least 7.0; (ii) the composition is free of EDAC and free of any amidine; (iii) the cocaine-succinyl-BSA is adsorbed onto a suitable adjuvant; and (iv) the cocaine-succinyl-BSA so adsorbed is suspended in a suitable buffer.

8. (canceled)

9. The composition of claim 7, wherein the average ratio of cocaine-succinyl moieties to BSA is selected from the group consisting of at least at least 7.5, at least 8.0, and at least 8.2.

10. The composition of claim 7, wherein the suitable adjuvant is aluminum hydroxide gel.

11. The composition of claim 7, wherein the suitable buffer is phosphate buffered saline.

12. The composition of claim 7, wherein the composition comprises a plurality of cocaine-succinyl-BSA, wherein (i) the average ratio of cocaine-succinyl moieties to BSA is at least 8.2; (ii) the composition is free of EDAC and free of any amidine; (iii) the cocaine-succinyl-BSA is adsorbed onto aluminum hydroxide gel; and (iv) the cocaine-succinyl-BSA so adsorbed is suspended in phosphate buffered saline.

13.-25. (canceled)

26. A method for making cocaine-succinyl-BSA, comprising the following steps:
(a) contacting cocaine with succinic anhydride in the presence of benzene under conditions permitting the formation of cocaine-succinate; and
(b) contacting the resulting cocaine-succinate with BSA under conditions permitting the formation of cocaine-succinyl-BSA, wherein (i) such conditions comprise the presence of tributylamine, dioxane, and isobutyl chloroformate, and (ii) neither step (a) nor step (b) is performed in the presence of EDAC or in the presence of any amidine.

27. (canceled)

28. A method for making cocaine-succinyl-BSA, comprising contacting cocaine-succinate with BSA under conditions permitting the formation of cocaine-succinyl-BSA, wherein (i) such conditions comprise the presence of tributylamine, dioxane, and isobutyl chloroformate, and (ii) the contacting is performed in the absence of EDAC and in the absence of any amidine.

29. (canceled)

30. An article of manufacture comprising (a) the composition of claim 7, and (b) a label indicating a use for the composition in treating a human subject addicted to cocaine.

31. An article of manufacture comprising (a) the composition of claim 12, and (b) a label indicating a use for the composition in treating a human subject addicted to cocaine.

32. An article of manufacture comprising (a) the composition of claim 7, and (b) a label indicating a use for the composition in inhibiting the onset of cocaine addiction in a non-addicted human subject.

33. An article of manufacture comprising (a) the composition of claim 12, and (b) a label indicating a use for the composition in inhibiting the onset of cocaine addiction in a non-addicted human subject.

34.-102. (canceled)
